SEQUENCE LISTING (2 sequences)

SEQ ID NO: 1 - DNA, 23 bp, Paralichthys olivaceus
  source 1..23: mol_type = genomic DNA
atgggtacgtcagtctggcctgg

SEQ ID NO: 2 - DNA, 23 bp, Takifugu rubripes
  source 1..23: mol_type = genomic DNA
agccggcggcgctgcagcccagg

(19) United States
(12) Patent Application Publication
(10) Pub. No.: US 2025/0143271 A1
(43) Pub. Date: May 8, 2025

(54) METHOD FOR PRODUCING STERILIZED MALE INDIVIDUALS OF MARINE FISH, METHOD FOR PREVENTING REPRODUCTION OF MARINE FISH, AND STERILIZED MALE INDIVIDUALS OF MARINE FISH

(71) Applicant: REGIONAL FISH INSTITUTE, LTD., Kyoto (JP)

(72) Inventor: Kenta KISHIMOTO, Kyoto (JP)

(73) Assignee: REGIONAL FISH INSTITUTE, LTD., Kyoto (JP)

(21) Appl. No.: 18/838,291

(22) PCT Filed: Jul. 5, 2023
(86) PCT No.: PCT/JP2023/024931

§ 371 Date: Aug. 14, 2024

(30) Foreign Application Priority Data
Jul. 8, 2022 (JP) 2022-110385

(51) Int. Cl. A01K 67/0276; C12N 9/22; C12N 15/11; C12N 15/113

(52) U.S. Cl. CPC A01K 67/0276; C12N 9/22; C12N 15/111; C12N 15/1138; A01K 2207/05; A01K 2217/058; A01K 2227/40; C12N 2310/141; C12N 2310/20; C12N 2310/531

(57) ABSTRACT

An object of the present invention is to produce sterilized male individuals of marine fish. Another object of the present invention is to provide a method for preventing reproduction using the sterilized male individuals. The objects are achieved by using a method for producing the sterilized male individuals of marine fish, the method including: suppressing functional expression of at least one selected from a follicle-stimulating hormone receptor (FSHR) and a ligand thereof.

### TECHNICAL FIELD

The present description discloses a method for producing sterilized male individuals, a method for preventing reproduction, and sterilized male individuals of marine fish.

### BACKGROUND ART

When reaching certain ages and body sizes, fish become sexually mature and engage in reproductive behavior to have offspring. This produces seedlings for aquaculture production. In the case where sexual maturation and reproduction of farmed fish remain in a natural state, fertilized eggs, seedlings, and adult fish sold to another vendor can be self-produced after a certain period of rearing, and such fish may engage in reproductive behavior with wild individuals for reproduction after escaping into the wild.

In addition, it has been studied to prevent self-production and reproduction in the environment by controlling sexual maturation and reproduction of bluegill as a designated invasive alien species by genetic mutation (Non Patent Literature 1).

It is possible to suppress sexual maturation and reproduction of one side of sexes by a genetic mutation while presence of primordial germ cells, oogonia, and spermatogonia, which are sources of gametes including eggs and sperm, is maintained. However, it is difficult to suppress sexual maturation and reproduction of both sexes simultaneously. For example, female medaka whose follicle-stimulating hormone receptor (FSHR) genes are knocked out (FSHR KO medaka) are found to be infertile. However, male individuals of FSHR KO medaka or sexually transformed male individuals can produce sperm normally and thus can reproduce (Non Patent Literature 2). The same phenomenon as that of FSHR KO medaka is also reported in zebrafish (Non Patent Literature 3).

### CITATION LIST

### Non Patent Literature

Non Patent Literature 1: https://www.erca.go.jp/suishinhi/seika/pdf/seika_1_h29/4-1408_2.pdf
Non Patent Literature 2: Murozumi et al. Endocrinology, August 2014, 155(8):3136-3145
Non Patent Literature 3: Endocrinology, October 2015, 156(10):3747-3762

### SUMMARY OF INVENTION

### Technical Problem

An object of the present invention is to produce sterilized male individuals of marine fish. Another object of the present invention is to provide a method for preventing reproduction using the sterilized male individuals.

### Solution to Problem

The present invention includes the following aspects.

### Item 1

A method for producing sterilized male individuals of marine fish, the method including: suppressing functional expression of at least one selected from a follicle-stimulating hormone receptor (FSHR) and a ligand thereof.

### Item 2

The method according to item 1, in which the functional expression is suppressed by introducing a loss-of-function mutation in at least one gene selected from the FSHR and the ligand thereof.

### Item 3

The method according to item 2, in which the loss-of-function mutation is introduced by at least one genome editing system selected from a clustered regularly interspaced short palindromic repeats/CRISPR associated protein 9 (CRISPR/Cas9) system, a Zinc-finger nuclease (ZFN) system, and a TAL effector nuclease (TALEN) system.

### Item 4

The method according to item 1, in which the functional expression is suppressed by suppressing expression of at least one selected from an FSHR gene and a ligand thereof.

### Item 5

The method according to item 4, in which the expression of the gene is suppressed by at least one type selected from a group including at least one type of an RNA molecule or a vector capable of expressing the RNA molecule selected from a group including a siRNA, a shRNA, and a miRNA targeting at least one mRNA selected from the FSHR and the ligand thereof.

### Item 6

The method according to item 1, in which the functional expression is suppressed by suppressing a function of at least one protein selected from the FSHR and the ligand thereof.

### Item 7

The method according to item 6, in which the function of the protein is suppressed by at least one type selected from a group including antibodies that specifically bind to at least one selected from the FSHR and the ligand thereof to suppress functional expression of a binding target by such binding.

### Item 8

A male individual of marine fish sterilized by suppressing functional expression of at least one selected from a follicle-stimulating hormone receptor (FSHR) and a ligand thereof.

### Item 9

A method for preventing reproduction of marine fish, the method including: introducing a male individual of marine fish, which is sterilized by suppressing functional expression of at least one selected from a follicle-stimulating hormone receptor (FSHR) and a ligand thereof, into an environment where a female individual of the marine fish is present or an environment where a female individual capable of mating with the marine fish is present, in which the female individual is not sterilized.

### Item 10

The method for preventing reproduction according to item 9 further including: also introducing a female individual of the marine fish sterilized by suppressing functional expression of at least one selected from the follicle-stimulating hormone receptor (FSHR) and the ligand thereof.

### Advantageous Effects of Invention

The present invention can provide the sterilized male individuals of marine fish. The present invention can also provide the method for preventing reproduction using these sterilized male individuals. The sterilized male individuals are expected to act as a deterrent against theft of farmed fish and reproduction of farmed fish by unauthorized persons using sold individuals.

### DESCRIPTION OF EMBODIMENTS

### 1. Method for Producing Sterilized Male Individuals of Marine Fish and Sterilized Male Individuals of Marine Fish

An embodiment of the present invention relates to a method for producing sterilized male individuals of marine fish.

In the present description, “marine fish” is not particularly limited. In the present description, marine fish can include saltwater fish, brackish water fish, diadromous fish, and the like.

The marine fish can include, for example, fishes of the families Paralichthys, Tetraodontidae: puffers, Ostraciidae: box fishes, Sparidae: sea breams and porgies, Serranidae: sea basses, Monacanthidae, Scombridae, Pleuronectidae, Carabidae, Lateolabrax, Moronidae, Rachycentridae, Cynoglossidae, Congridae, and the like.

Brackish water fish include, for example, fishes of the family Latidae and the like.

Diadromous fish can include fishes of the families Salmonidae, Osmeridae, Anguillidae, and the like.

Fishes of the family Paralichthys can include, for example, Paralichthys olivaceus (TEMMINCK et SCHLEGEL), fishes belonging to the genus Pseudorhombus (ocellated flounder (Pseudorhombus dupliciocellatus), roughscale flounder (Pseudorhombus oligodon), Pseudorhombus ctenosquamis, largetooth flounder (Pseudorhombus arsius), fivespot flounder (Pseudorhombus pentophthalmus), cinnamon flounder (Pseudorhombus cinnamoneus), central spotted flounder (Pseudorhombus levisquamis), and the like), small flounder (Tarphops oligolepis) and the like belonging to the genus Tarphops, intermediate flounder (Asterorhombus intermedius) belonging to the genus Asterorhombus, dusky sole (Lepidopsetta mochigarei) belonging to the genus Lepidopsetta, Indo-Pacific ocellated flounder (Taeniopsetta ocellata) belonging to the genus Taeniopsetta, and the like. A preferred fish of the family Paralichthys is Paralichthys olivaceus (TEMMINCK et SCHLEGEL).

Fishes of the family Tetraodontidae can include, for example, fishes belonging to the genus Takifugu (torafugu (Takifugu rubripes), purple puffer (Takifugu porphyreus), grass puffer (Takifugu niphobles), and the like), half-smooth golden pufferfish (Lagocephalus wheeleri) and the like belonging to the genus Lagocephalus, and the like. A preferred fish of the family Tetraodontidae is torafugu (Takifugu rubripes).

Fishes of the family Ostraciidae can include, for example, bluespotted boxfish (Ostracion immaculatus) and the like belonging to the genus Ostracion.

Fishes of the family Sparidae can include, for example, fishes belonging to the genus Pagrus (red seabream (Pagrus major), squirefish (Pagrus auratus), and the like), fishes belonging to the genus Acanthopagrus (black porgy (Acanthopagrus schlegeli), yellowfin seabream (Acanthopagrus latus), and the like), yellowback seabream (Dentex hypselosomus) and the like belonging to the genus Dentex, and gilthead seabream (Sparus aurata) and the like belonging to the genus Sparus.

Fishes of the family Serranidae can include, for example, fishes belonging to the genus Epinephelus (convict grouper (Epinephelus eptemfasciatus), longtooth grouper (Epinephelus bruneus), Hong Kong grouper (Epinephelus akaara), Malabar grouper (Epinephelus malabaricus), white grouper (Epinephelus aeneus), banded grouper (Epinephelus amblycephalus), areolate grouper (Epinephelus areolatus), duskytail grouper (Epinephelus bleekeri), pale margin grouper (Epinephelus bontoides), brown spotted grouper (Epinephelus chlorostigma), orange-spotted grouper (Epinephelus coioides), blacktip grouper (Epinephelus fasciatus), brown-marbled grouper (Epinephelus fuscoguttatus), starry grouper (Epinephelus labriformis), giant grouper (Epinephelus lanceolatus), highfin grouper (Epinephelus maculatus), dusky grouper (Epinephelus marginatus), white-streaked grouper (Epinephelus ongus), camouflage grouper (Epinephelus polyphekadion), longfin grouper (Epinephelus quoyanus), sixbar grouper (Epinephelus sexfasciatus), Nassau grouper (Epinephelus striatus), greasy grouper (Epinephelus tauvina), potato grouper (Epinephelus tukula), and the like), humpback grouper (Cromileptes altivelis) and the like belonging to the genus Cromileptes, leopard coralgrouper (Plectropomus leopardus) and the like belonging to the genus Plectropomus, and crossbreds among fishes of the family Serranidae.

Fishes of the family Monacanthidae can include, for example, thread-sail filefish (Stephanolepis cirrhifer) and the like belonging to the genus Stephanolepis, and black scraper (Thamnaconus modestus) and the like belonging to the genus Thamnaconus.

Fishes of the family Scombridae can include, for example, fishes belonging to the genus Scombrini (chub mackerel (Scomber japonicus), Atlantic mackerel (Scomber scombrus), blue mackerel (Scomber australasicus), and the like), fishes belonging to the genus Thunnini (Pacific bluefin tuna (Thunnus orientalis), Atlantic bluefin tuna (Thunnus thynnus), southern bluefin tuna (Thunnus maccoyii), bigeye tuna (Thunnus obesus), yellowfin tuna (Thunnus albacares), albacore (Thunnus alalunga), longtail tuna (Thunnus tonggol), and the like), fishes belonging to the genus Euthynnus (eastern little tuna (Euthynnus affinis), little tunny (Euthynnus alletteratus), and the like), skipjack tuna (Katsuwonus pelamis) and the like belonging to the genus Katsuwonus, fishes belonging to the genus Scomberomorini, fishes belonging to the genus Auxis, fishes belonging to the genus Sardini, fishes belonging to the genus Gymnosarda, and the like.

Fishes of the family Pleuronectidae can include, for example, littlemouth flounder (Pseudopleuronectes herzensteini), marbled flounder (Pleuronectes yokohamae), stone flounder (Kareius bicoloratus), Pacific halibut (Hippoglossus stenolepis), barfin flounder (Verasper moseri), and the like.

Fishes of the family Carangidae can include, for example, fishes belonging to the genus Seriola (greater amberjack (Seriola dumerili), yellowtail amberjack (Seriola lalandi), almaco jack (Seriola rivoliana), Japanese amberjack (Seriola quinqueradiata), and the like), Japanese jack mackerel (Trachurus japonicus), hard-tail jack (Pseudocaranx dentex), and the like belonging to the genus Pseudocaranx, and snubnose pompano (Trachinotus blochii) and the like belonging to the genus Trachinotus.

Fishes of the family Lateolabrax can include, for example, blackfin seabass (Lateolabrax latus), spotted sea bass (Lateolabrax maculatus), and the like.

Fishes of the family Moronidae can include, for example, European seabass (Dicentrarchus labrax) and the like.

Fishes of the family Rachycentridae can include, for example, cobia (Rachycentron canadum) and the like.

Fishes of the family Cynoglossidae can include, for example, red tonguesole (Cynoglossus joyneri), tongue sole (Cynoglossus semilaevis), and the like.

Fishes of the family Congridae can include, for example, whitespotted conger (Conger myriaster), Beach conger (Conger japonicus), and the like.

Fishes of the family Latidae can include, for example, fishes of the genus Lates such as barramundi perch (Lates calcarifer) and Nile perch (Lates niloticus).

Fishes of the family Salmonidae can include, for example, fishes belonging to the genus Oncorhynchus (rainbow trout (Oncorhynchus mykiss), Chinook salmon (Oncorhynchus tshawytscha), cherry salmon (Oncorhynchus masou masou), red-spotted masu salmon (Oncorhynchus masou ishikawae), black kokanee (Oncorhynchus kawamurae), pink salmon (Oncorhynchus gorbuscha), chum salmon (Oncorhynchus keta), sockeye salmon (Oncorhynchus nerka), coho salmon (Oncorhynchus kisutch), and the like), fishes belonging to the genus Salmo (brown trout (Salmo trutta), Atlantic salmon (Salmo salar), and the like), fishes belonging to the genus Salvelinus (Dolly Varden (Salvelinus malma), Arctic char (Salvelinus alpinus), whitespotted char (Salvelinus leucomaenis), brook trout (Salvelinus fontinalis), lake trout (Salvelinus namaycush), and the like), and Sakhalin taimen (Parahucho perryi) and the like belonging to the genus Hucho.

Fishes of the family Osmeridae can include, for example, ayu (Plecoglossus altivelis) and the like belonging to the genus Plecoglossinae, fishes belonging to the genus Hypomesinae (Japanese smelt (Hypomesus nipponensis), Japanese surfsmelt (Hypomesus japonicus), and the like), and fishes belonging to the genus Osmerinae (Arctic rainbow smelt (Osmerus mordax dentex), Shishamo smelt (Spirinchus lanceolatus), Japanese icefish (Salangichthys microdon), and the like).

Fishes of the family Anguillidae can include, for example, Japanese eel (Anguilla japonica), European eel (Anguilla anguilla), and the like.

In the present description, the fish may be true breds or crossbreds. The crossbreds can include, for example, hybrids derived from intergeneric crossing.

The “fish” is preferably farmed fish. In addition, the “farmed fish” can include, for example, fish that are bred for purposes of food, reproduction, decoration, and the like.

In the present description, “sterility” is intended to mean failure to form a gamete at reproductive age of months or functional failure of the gamete even when the gamete is formed.

In the present description, the method for producing the sterilized male individuals of marine fish includes a step of suppressing functional expression of at least one selected from a follicle-stimulating hormone receptor (FSHR) and a ligand thereof. The ligands may include gonadotropins such as a glycoprotein hormone (GP) and a follicle-stimulating hormone (FSH).

For example, an mRNA sequence of the FSHR of bastard halibut (Paralichthys olivaceus) is registered as NCBI Reference Sequence: XM_020083600. An mRNA sequence of the GP of Paralichthys olivaceus is registered in GenBank: XM_020099391. An mRNA sequence of the FSH of Paralichthys olivaceus is registered in GenBank: XM_020084778. For example, an mRNA sequence of the FSHR of Takifugu rubripes is registered as NCBI Reference Sequence: MK359809. An mRNA sequence of the GP of Takifugu rubripes is registered in GenBank: DAA06175. An mRNA sequence of the FSH of Takifugu rubripes is registered in GenBank: DAA06176. Specific examples include the FSHR genes shown in Table 1 below, the GPs shown in Table 2 below, and the FSHs shown in Table 3.

TABLE 1

| | | GenBank ID | Gene ID |
|---|---|---|---|
| Species Name | Scientific Name | (NCBI) | (NCBI) |
| Torafugu | Takifugu rubripes | MK359809 | |
| Bastard halibut | Paralichthys olivaceus | XM_020083600 | 109627163 |
| Gilthead seabream | Sparus aurata | XM_030399424 | 115570729 |
| Atlantic cod | Gadus morhua | XM_030341011 | 115531650 |
| Rainbow trout | Oncorhynchus mykiss | NM_001124327 | 100135990 |
| Coho salmon | Oncorhynchus kisutch | XM_031805247 | 109870448 |
| Sockeye salmon | Oncorhynchus nerka | XM_029684751 | 115144066 |
| Chinook salmon | Oncorhynchus | XM_042305990 | 112224765 |
| | tshawytscha | | |
| Brown trout | Salmo trutta | XM_029764582 | 115201179 |
| Arctic char | Salvelinus alpinus | XM_023993153 | 111967791 |
| Atlantic salmon | Salmo salar | NM_001126230 | 100135779 |
| Giant grouper | Epinephelus | XM_033611329 | 117247103 |
| | lanceolatus | | |
| Orange-spotted | Epinephelus coioides | HQ650769 | |
| grouper | | | |
| Tongue sole | Cynoglossus | NM_001294190 | 103381541 |
| | semilaevis | | |
| Chub mackerel | Scomber japonicus | KC207943 | |
| Greater amberjack | Seriola dumerili | XM_022743559 | 111220462 |
| Yellowtail | Seriola lalandi | XM_0234406569 | 111655238 |
| amberjack | | | |
| Japanese seabass | lateolabrax japonicus | JN625218 | |
| European seabass | Dicentrarchus labrax | AY642113 | |
| European eel | Anguilla anguilla | XM_035405941 | 118221147 |

TABLE 2

| | | GenBank ID | Gene ID |
|---|---|---|---|
| Species Name | Binominal Name | (NCBI) | (NCBI) |
| Atlantic bluefin tuna | Thunnus thynnus | | |
| Convict grouper | Epinephelus | BAC78811 | |
| | septemfasciatus | | |
| Longtooth grouper | Epinephelus bruneus | ABQ57398 | |
| Striped sea-bass | Morone saxatilis | Q91119 | |
| European seabass | Dicentrarchus labrax | AAK49431 | |
| Red seabream | Pagrus major | BAB18562 | |
| Black porgy | Acanthopagrus schlegelii | ABQ96863 | |
| Grass puffer | Takifugu niphobles | BAJ12079 | |
| Torafugu | Takifugu rubripes | DAA06175 | |
| Japanese flounder | Paralichthys olivaceus | XM_020084778 | 109627943 |
| Atlantic halibut | Hippoglossus | CAD10503 | |
| | hippoglossus | | |
| Senegalese sole | Solea senegalensis | ABW81405 | |
| Mummichog | Fundulus heteroclitus | AAB60605 | |
| Coho salmon | Oncorhynchus kisutch | AA072301 | |
| Chum salmon | Oncorhynchus keta | AAA49404 | |
| Japanese eel | Anguilla japonica | BAD14301 | |
| European eel | Anguilla anguilla | P27794 | |

TABLE 3

| | | GenBank ID | Gene ID |
|---|---|---|---|
| Species Name | Binominal Name | (NCBI) | (NCBI) |
| Atlantic bluefin tuna | Thunnus thynnus | ABP04057 | |
| Convict grouper | Epinephelus | BAC78812 | |
| | septemfasciatus | | |
| Longtooth grouper | Epinephelus bruneus | ABQ57399 | |
| Striped sea-bass | Morone saxatilis | Q91120 | |
| European seabass | Dicentrarchus labrax | AAN40506 | |
| Red seabream | Pagrus major | BAB18563 | |
| Black porgy | Acanthopagrus schlegelii | AAX18926 | |
| Grass puffer | Takifugu niphobles | BAJ12081 | |
| Torafugu | Takifugu rubripes | DAA06176 | |
| Japanese flounder | Paralichthys olivaceus | XM_020099391 | 109637185 |
| Atlantic halibut | Hippoglossus | CAD10501 | |
| | hippoglossus | | |
| Senegalese sole | Solea senegalensis | ABW81403 | |
| Mummichog | Fundulus heteroclitus | P30971 | |
| Coho salmon | Oncorhynchus kisutch | AA072299 | |
| Chum salmon | Oncorhynchus keta | P10257 | |
| Japanese eel | Anguilla japonica | Q9YGK3 | |

| | | |
|---|---|---|
| European eel | Anguilla anguilla | AAN73407 |

The method for suppressing the functional expression of at least one selected from the FSHR and the ligand thereof is not limited. For example, the functional expression may be suppressed by introducing the loss-of-function mutation in at least one selected from the FSHR and the ligand thereof, suppressing expression of at least one gene selected from the FSHR and the ligand thereof, or neutralizing a function of at least one protein selected from the FSHR and the ligand thereof.

A method for introducing the loss-of-function mutation in at least one selected from the FSHR and the ligand thereof is not limited as long as the loss-of-function mutation can be introduced into a target gene in a genome of target fish. Such a method is known. Examples of the method for introducing the loss-of-function mutation can include site-directed mutagenesis methods, such as a genome editing method and a homologous recombination method, and a random mutagenesis method.

The genome editing method can include a method for introducing a protein and a nucleic acid, which constitute a genome editing technique, or vectors encoding these protein and nucleic acid. Examples of the protein can include clustered regularly interspaced short palindromic repeats (CRISPR) enzymes. More specifically, examples of the CRISPR enzymes can include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. Examples of the nucleic acids can include crRNA, tracrRNA, and a single-stranded nucleic acid in which these are linked via a linker. In this case, for example, the nucleic acid is designed, for example, such that a nucleotide sequence to anneal to a target sequence in the crRNA is complementary to a nucleotide sequence encoding at least one selected from the FSHR and the ligand thereof. A single type of the nucleic acid may be used alone, or two or more types of the nucleic acids may be used in combination. The genome editing systems using the CRISPR enzyme can include a clustered regularly interspaced short palindromic repeats/CRISPR associated protein 9 (CRISPR/Cas9) system. As another genome editing system, at least one method can be selected from a Zinc-finger nuclease (ZFN) system and a TAL effector nuclease (TALEN) system. Preferably, the genome editing system is the CRISPR/Cas9 system that introduces Cas9 as mRNA or a protein and introduces gRNA as sgRNA or crRNA and tracrRNA. In a vector-based CRISPR/Cas9 system, the nucleic acid encoding CRISPR and the nucleic acid encoding Cas9 may be on different vectors or on one vector. A promoter for the CRISPR function is not particularly limited. However, a U6 promoter is preferred. A promoter for the Cas9 function is not particularly limited. However, a promoter expressed in a mammalian cell, such as a Cytomegalovirus promoter, is preferred. Preferably, a commercially available vector such as a pX330-U6-Chimeric_BB-CBh-hSpCas9 vector can be used as the CRISPR/Cas9 system.

A sequence that targets the FSHR gene or a gonadotropin gene incorporated into a CRISPR sequence (hereinafter also referred to as a “target sequence”) is not particularly limited as long as such a sequence can be transcribed by the CRISPR/Cas9 system into guide RNA (also referred to as gRNA, sgRNA, or crRNA) or introduced, as the guide RNA that contains the complementary sequence to the target sequence, into cells to recombine the FSHR gene or the gonadotropin gene. In general, it is said that, as the target sequence, a sequence of about 20 bases in a 5′ upstream region of a nucleotide sequence “NGG” (a PAM sequence: N is any of nucleotide A, G, T, C), which is present in the FSHR gene or the gonadotropin gene, can be selected. The target sequence can be designed by using any of known design tools published by an Optimized CRISPR design tool (a website of Zhang Lab at Massachusetts Institute of Technology (http://crispr.mit.edu/)), E-CRISP (http://www.e-crisp.org/E-CRISP/(German Cancer Research Center)), ZiFiT Targeter (http://zifit.partners.org/ZiFit/(Zing Finder Consortium)), Cas9 design (http://cas9.cbi.pku.edu.cn (Peking University)), CRISPRdirect (http://crispr.dbcls.jp (the University of Tokyo)), CRISPR-P (http://cbi.hzau.edu.cn/crispr/(Huazhong Agricultural University)), CRISPR RGEN Tools (http://www.rgenome.net/(Seoul National University)), and the like.

In a case of bastard halibut (Paralichthys olivaceus), examples of a sequence targeting the FSHR gene can include 5′-ATGGGTACGTCAGTCTGGCCTGG-3′ (SEQ ID NO: 1). In a case of torafugu (Takifugu rubripes), examples of a sequence targeting the FSHR gene can include 5′-AGCCGGCGGCGCTGCAGCCCAGG-3′ (SEQ ID NO: 2).

In addition, preferably, when single nucleotide polymorphisms (SNPs) exist in the PAM sequence, such a sequence is preferably avoided. In regard to the target sequence, when the SNPs of an individual are known, the sequence is preferably optimized for each of the SNPs.

In the 5′-terminal region of the target sequence, the sequence may be one, two, three, or four bases shorter, preferably, one, two, or three bases shorter.

In the CRISPR/Cas9 system, a gene, as a vector, may be introduced into cells, or a combination of a gRNA, a crRNA, a trans-activating crRNA (tracrRNA), and an RNA encoding Cas9 synthesized by artificial synthesis or in vitro transcription may be introduced into the cells. Alternatively, a combination of a Cas9 protein and a guide RNA may be introduced into the cell.

Furthermore, in the genome editing system, a donor oligo DNA such as a single-stranded oligos (ssODNs) may be co-introduced into the cell. The ssODNs can be designed according to a known method.

The genome editing system can perform microinjection into cytoplasm of a fertilized egg, preferably, a fertilized egg at a one-cell stage. For example, when being introduced, the Cas9 protein can be injected in a range of 5 pg to 100 pg, preferably 10 pg to 80 pg, more preferably 10 pg to 50 pg per fertilized egg. At this time, the guide RNA can be injected in a range of 0.1 pg to 50 pg, preferably 0.5 pg to 20 pg, more preferably 1 pg to 5 pg.

Example 1 described below can be referred for a method for introducing a mutation using the genome editing technique, for example.

Examples of the random mutagenesis method can include: treatment with radiation such as α-rays, β-rays, γ-rays, and X-rays; a chemical substance treatment using a mutagenic agent such as ethyl methanesulfonate (EMS) or ethynylnitrosourea (ENU); and a heavy ion beam treatment.

A method for suppressing expression of at least one gene selected from the FSHR and the ligand thereof can include a method for introducing at least one type selected from a group including RNA molecules targeting at least one mRNA selected from the FSHR and the ligand thereof or vectors capable of expressing such RNA molecules. The “RNA molecules targeting at least one mRNA selected from the FSHR and the ligand thereof” are not limited as long as the RNA molecules target at least one mRNA selected from the FSHR and the ligand thereof and can suppress expression of such an FSHR or ligand thereof. Examples of the RNA molecules can include those having a degrading action on the target mRNA such as a siRNA, a shRNA, a dsRNA, a miRNA and/or those suppressing mRNA translation. A person skilled in the art can design sequences of these RNA molecules appropriately using a known method on the basis of information on a nucleotide sequence of the target gene. The RNA molecules may be prepared on the basis of a known method, or the RNA molecules available in the market may be obtained and used. As the RNA molecules, the siRNA, the shRNA, and the miRNA are preferred, and the siRNA and the shRNA are especially preferred. The vectors capable of expressing the RNA molecules targeting the mRNA of the FSHR or the mRNA of the gonadotropin are not particularly limited as long as the vectors can express the RNA molecules suppressing expression of the FSHR protein or the gonadotropin protein in an individual body or in a cell. An example of such a vector can be a hairpin RNA expression vector. The hairpin RNA expression vectors at least include: a sense-strand DNA nucleotide sequence having the same sequence as a sense strand of the target mRNA (however, uracil of the mRNA is replaced to thymine) on a downstream of a promoter sequence such as a U6 promoter; a loop nucleotide sequence that forms a loop structure after transcription; an antisense-strand DNA sequence that can complementarily bind in whole or in part to the sense-strand DNA nucleotide sequence; and a terminator sequence. Examples of the vectors can include a plasmid vector, an adenovirus vector, a retrovirus vector, and a lentivirus vector.

At least one RNA molecule selected from the group including the siRNA, the shRNA, and the miRNA can be injected by microinjection in a range of 5 pg to 100 pg, preferably 10 pg to 80 pg, more preferably 10 pg to 50 pg per fertilized egg. At least one RNA molecule selected from the group including the siRNA, the shRNA, and the miRNA, or the vector capable of expressing such an RNA molecule can be injected by microinjection in a range of 5 pg to 100 pg, preferably 10 pg to 80 pg, more preferably 10 pg to 50 pg per fertilized egg. The vector can be linearized when necessary.

The function of the protein can be suppressed using at least one type selected from a group including antibodies that specifically bind to the FSHR protein or the gonadotropin protein to suppress functional expression of the binding target by such binding. Such an antibody may be a polyclonal antibody or a monoclonal antibody. A person skilled in the art can appropriately prepare both the polyclonal antibody and the monoclonal antibody using a known method. In addition, the antibody may be an antibody fragment such as Fab, F(ab)2, a diabody, scFv, a minibody, a peptibody, or a mimetibody.

The antibody can be injected in a range of 5 pg to 100 pg, preferably 10 pg to 80 pg, more preferably 10 pg to 50 pg per fertilized egg.

Hatchlings hatched from fertilized eggs and fries are reared for one to five months, and an individual in which a mutation is induced in the FSHR gene or the gonadotropin gene is selected by a mutation analysis method, such as a heteroduplex mobility analysis, Cel-1 assay, or a T7 endonuclease assay sequence analysis. A male individual can be selected by confirming spermatogenesis or ovulation through abdominal pressure, by inserting a tube through a cloaca and extracting a portion of a gonad to confirm the sex of a germ cell, or by identifying the sex by a sex-determining gene or a sex chromosome genotype. The male individual is reared at a water temperature of about 15.0 to 22.0° C. from 5 to 16 months of age. Daylight hours can be set under a natural condition. Other rearing conditions can be set under general aquaculture conditions. The same food as that generally fed under the general aquaculture conditions can be used.

For example, in the case of bastard halibut (Paralichthys olivaceus), hatchlings hatched from fertilized eggs and fries are reared at the water temperature of about 14.4 to 16.4° C. until 2.6 months of age. Individuals with the induced mutation in the FSHR gene are selected by the heteroduplex mobility analysis. The individuals are reared at the water temperature of about 16 to 19.3° C. until 5.8 months of age, and then reared at the water temperature of about 14.3 to 20.6° C. until 14.7 months of age. The individuals are reared at the water temperature of about 15.6 to 22.2° C. from 14.7 months of age. The daylight hours can be set under the natural condition. The other rearing conditions can be set under the general aquaculture conditions. The same food as that generally fed under the general aquaculture conditions can be used.

The above method can be used to obtain the sterilized male individuals of marine fish (an F0 generation in this case). In addition, female individuals from the F0 generation onward can be mated with the male individuals from the F0 generation onward to obtain the sterilized male individuals of marine fish (F1 and later generations). Alternatively, the sterilized male individuals of marine fish (the F1 and later generations) can be obtained by mating any male individuals with female individuals from the F0 generation onward for gynogenesis. Further alternatively, the sterilized male individuals of marine fish (the F1 and later generations) can be obtained by mating the male individuals of the F0 generation onward with any female individuals for androgenesis.

Moreover, the male individuals may be obtained through sexual transformation of the female individuals obtained by the above method. Examples of a method for the sexual transformation include rearing at the water temperature of 25° C. or higher from ages 0 to 3 months of age, administering a male sex hormone (an androgen), such as testosterone or 11-ketotestosterone, or an aromatase inhibitor, or the like.

### 2. Method for Preventing Reproduction of Marine Fish

An embodiment of the present invention includes introducing the sterilized male individuals of marine fish produced by the method described in above Section 1 into an environment where the female individuals of the marine fish are present or an environment where the female individuals of marine fish capable of mating with the marine fish are present. For example, torafugus (Takifugu rubripes) can be mated with fishes of the family Tetraodontidae such as fishes of the genus Takifugu including grass puffer (Takifugu niphobles) and purple puffer (Takifugu porphyreus).

The environment may be a captive environment or a natural environment.

The female individuals may or may not be sterilized.

In the method for preventing reproduction, the female individuals capable of mating with the male individuals produced according to the method described in above Section 1 may be introduced into the environment together with the sterilized male individuals of marine fish.

### EXAMPLES

An aspect of the present invention will be described in detail in Examples below. However, the present invention should not be construed as being limited to Examples.

1. Production of Bastard Halibut (Paralichthys olivaceus) Deficient in FSHR Gene and Observation of Sexual Maturation Period of Males

Bastard halibut (Paralichthys olivaceus) deficient in a portion of the FSHR gene were produced and compared with wild bastard halibut (Paralichthys olivaceus) to examine suppression of male sexual maturity.

### (1) Preparation of Fertilized Eggs

Eggs (unfertilized eggs) and sperm were respectively collected from sexually mature male and female bastard halibut (Paralichthys olivaceus) through abdominal pressure. The obtained eggs and sperm were artificially inseminated to obtain fertilized eggs.

### (2) Introduction of Mutation

A mutation was introduced using the Cas9 protein and the guide RNA. The sequence of the target gene was 5′-ATGGGTACGTCAGTCTGGCCTGG-3′ (SEQ ID NO: 1), and the RNA was synthesized using CUGA®7 gRNA Synthesis Kit available from NIPPON GENE CO., LTD.

The mutation was introduced into the FSHR gene by microinjection of a solution, which was prepared to have 10 to 50 pg of the Cas9 protein and 1 to 5 μg of the guide RNA, into cytoplasm of a fertilized egg at the one-cell stage obtained in (1) above. The fertilized eggs transfected with the Cas9 protein and the guide RNA were reared and hatched under normal rearing conditions to obtain an F0 generation.

(3) Observation of Sexual Maturation in Male Bastard Halibut (Paralichthys olivaceus) Deficient in FSHR Gene

The introduction of the above mutation resulted in lack of gonad development, lack of spermiation, or lack of gametogenesis in both sexes at 8 to 12 months of age.

Presence or absence of spermiation, presence or absence of sperm in the gonad, and sperm motility in male bastard halibut (Paralichthys olivaceus) genome-edited for the FSHR gene were evaluated by the following methods.

The mutated individuals were reared for one month at the water temperature of 16° C., at which spermiation by bastard halibut (Paralichthys olivaceus) was considered to occur.

The individuals at 8 months of age were subjected to abdominal pressure to check the presence or absence of spermiation.

Thereafter, the individuals were dissected to weigh the gonads and check the presence or absence of sperm in testes.

The obtained sperm was evaluated for normality by morphology and motility by exposure to seawater. Table 4 shows evaluation results of the individuals at 8 months of age.

TABLE 4

| | | Total | Body | Gonad | | | | |
|---|---|---|---|---|---|---|---|---|
| Individual | Genome- | length | weight | weight | | | Sperm in | Sperm |
| number | editing | (cm) | (g) | (g) | GSI*³ | Spermiation | gonad | motility |
| 1 | GE*¹ | 24.5 | 170.5 | 0.371 | 0.22 | Absent | Absent | — |
| 2 | GE*¹ | 24.0 | 170.6 | 0.484 | 0.19 | Absent | Absent | — |
| 3 | GE*¹ | 23.5 | 147.0 | 0.596 | 0.41 | Absent | Absent | — |
| 4 | WT*² | 27.0 | 214.0 | 0.643 | 0.30 | Absent | Present | Present |

*¹Genome-edited
*²Not genome-edited
*³GSI = gonad weight/body weight × 100

In the male bastard halibut (Paralichthys olivaceus) genome-edited for the FSHR gene, the spermiation was not observed, and sperm was not present in the gonads.

Similarly, it was confirmed that spermiation did not occur in two genome-edited individuals at 12 months of age (weighed 438 g and 351 g) and gonads thereof were atrophied.

2. Production of torafugu (Takifugu rubripes) Deficient in FSHR Gene and Observation of Sexual Maturation Period of Males

torafugu (Takifugu rubripes) deficient in a portion of the FSHR gene were produced and compared with wild torafugu (Takifugu rubripes) to examine suppression of male sexual maturity.

### (1) Preparation of Fertilized Eggs

Eggs (unfertilized eggs) and sperm were respectively collected from sexually mature male and female torafugu (Takifugu rubripes) through abdominal pressure. The obtained eggs and sperm were artificially inseminated to obtain the fertilized eggs.

### (2) Introduction of Mutation

A mutation was introduced using the Cas9 mRNA or protein and the guide RNA. The sequence of the target gene was 5′-AGCCGGCGGCGCTGCAGCCCAGG-3′ (SEQ ID NO: 2). The mutation was introduced into the FSHR gene by microinjection of a solution, which was prepared to have 2 to 10 μg of the Cas9 mRNA or protein and 1 to 5 μg of the guide RNA, into cytoplasm of a fertilized egg at the one-cell stage. The fertilized eggs transfected with the Cas9 mRNA or protein and the guide RNA were reared and hatched under normal rearing conditions to obtain an F0 generation.

(3) Observation of Sexual Maturation in Male torafugu (Takifugu rubripes) Deficient in FSHR Gene

The introduction of the above mutation resulted in lack of gonad development, lack of spermiation, or lack of gametogenesis in both sexes at 12 to 24 months of age.

Sequence ListingP23-141WO_PCT_Sterilized Male_Individuals of_Marine Fish 20230705_103904_4.xml

1. A method for producing sterilized male individuals of marine fish, the method comprising:
suppressing functional expression of at least one selected from a follicle-stimulating hormone receptor (FSHR) and a ligand thereof.

2. The method according to claim 1, wherein
the functional expression is suppressed by introducing a loss-of-function mutation in at least one gene selected from the FSHR and the ligand thereof.

3. The method according to claim 2, wherein
the loss-of-function mutation is introduced by at least one genome editing system selected from a clustered regularly interspaced short palindromic repeats/CRISPR associated protein 9 (CRISPR/Cas9) system, a Zinc-finger nuclease (ZFN) system, and a TAL effector nuclease (TALEN) system.

4. The method according to claim 1, wherein
the functional expression is suppressed by suppressing expression of at least one selected from the FSHR gene and the ligand thereof.

5. The method according to claim 4, wherein
the expression of the gene is suppressed by at least one type selected from a group including at least one type of an RNA molecule or a vector capable of expressing the RNA molecule selected from a group including a SiRNA, a shRNA, and a miRNA targeting at least one mRNA selected from the FSHR and the ligand thereof.

6. The method according to claim 1, wherein
the functional expression is suppressed by suppressing a function of at least one protein selected from the FSHR and the ligand thereof.

7. The method according to claim 6, wherein
the function of the protein is suppressed by at least one type selected from a group including antibodies that specifically bind to at least one selected from the FSHR and the ligand thereof to suppress functional expression of a binding target by such binding.

8. A male individual of marine fish sterilized by suppressing functional expression of at least one selected from a follicle-stimulating hormone receptor (FSHR) and a ligand thereof.

9. A method for preventing reproduction of marine fish, the method comprising:
introducing a male individual of marine fish, which is sterilized by suppressing functional expression of at least one selected from a follicle-stimulating hormone receptor (FSHR) and a ligand thereof, into an environment where a female individual of the marine fish is present or an environment where a female individual capable of mating with the marine fish is present, wherein
the female individual is not sterilized.

10. The method for preventing reproduction according to claim 9 further comprising:
also introducing a female individual of the marine fish sterilized by suppressing functional expression of at least one selected from the follicle-stimulating hormone receptor (FSHR) and the ligand thereof.
